(19) United States
(12) Patent Application Publication
(10) Pub. No.: US 2012/0198929 A1
(43) Pub. Date: Aug. 9, 2012

(54) METHOD OF DETERMINING WHEN A PERSON HAS REACHED A MINIMUM LEVEL OF PHYSICAL FITNESS

(76) Inventor: Ramon Rudolph De Hoyos, Melbourne, FL (US)

(21) Appl. No.: 13/252,252

(22) Filed: Oct. 4, 2011

Related U.S. Application Data
(60) Provisional application No. 61/441,192, filed on Feb. 9, 2011.

(51) Int. Cl. A61B 5/22

(52) U.S. Cl. 73/379.01

(57) ABSTRACT

The method disclosed is used in determining who is truly fit and is a capable low crouch duck walker. It establishes a distance of about 100 yards more or less as a minimum distance to cover without standing up or touching the ground with ones hands while in the low crouch duck walk.

### REFERENCE TO RELATED APPLICATIONS

This patent application claims the benefit of U.S. Provisional Application No. 61/441,192 filed on Feb. 9, 2011, the disclosure of which is incorporated herein by reference in its entirety.

### BACKGROUND OF THE INVENTION

1. Field of the Invention

The present invention relates generally to determining the fitness of a person and more specifically to a method of determining when a person has achieved a minimum level of physical fitness without using an exercise machine or an electronic measuring device such as a pulse measuring monitor.

2. Description of Related Art

Methods and apparatus for identifying a physical fitness level of a person is known in the prior art. More specifically, by way of example, U.S. PreGrant Publication No. 2010/0249619 to KASAMA discloses an apparatus for determining physical fitness levels, includes: a pulse measuring unit which measures a pulse rate of a user at rest; a measurement processor which acquires a pulse rate ratio of the pulse rate relative to a maximum pulse rate which is determined by an actual age of the user, estimates an oxygen uptake ratio on the basis of the pulse rate ratio and the actual age, and calculates a maximum oxygen uptake from an oxygen uptake at rest by using the oxygen uptake ratio.

U.S. PreGrant Publication No. 2005/0172975 to Downs discloses a method of exercising an exercise participant comprises the steps of first providing a participant with an activity chart so that the participant can determine a fitness level, then assigning a daily cardio exercise fitness number to the participant that is based on the participant's fitness level, wherein the cardio exercise fitness number will determine what cardio exercises the participant is to perform, then assigning a daily resistance exercise fitness number to the participant that is based on the participant's fitness level, wherein the resistance exercise fitness number will determine what resistance exercises the participant is to perform.

U.S. PreGrant Publication No. 2005/0164857 to Black discloses a plurality of fitness cards arranged in a flexible and resilient card holder which provide a user with a method for managing fitness activities. Each of the fitness cards includes a fitness activity description that may include any combination of fitness activity identifiers, images, and instructions.

U.S. Pat. No. 7,837,546 to Astilean discloses a portable, electronic device having a memory, an operating program, a user interface, a display and a location signal receiver adapted to receive location signals from a plurality of sources external to the fitness training device. A command and control executes the program and generates control signals based upon the user entered data and the location signals from the receiver.

U.S. Pat. No. 6,783,482 to Oglesby, et al. discloses a microprocessor based exercise treadmill control system which includes programs operative to: permit a set of user controls to cause the treadmill to initially operate at predetermined speeds; permit the user to design custom workouts; and permit the user to switch between workout programs while the treadmill is in operation.

U.S. Pat. No. 6,736,759 to Stubbs, et al. discloses an exercise monitoring system which includes an electronic positioning device; a physiological monitor; and a display unit configured for displaying data provided by the electronic positioning device and the physiological monitor

U.S. Pat. No. 4,566,461 to Lubell, et al. discloses a heart rate monitor device suitable for use in monitoring aerobic exercise training, automatically calculates a fitness parameter for a subject by monitoring the subject's heart rate and pacing the subject through a submaximal exercise stress test protocol.

U.S. Pat. No. 4,367,752 to Jimenez, et al. discloses a transducer mounted on the subject for monitoring the quantity of repetitive actions of a limb.

### SUMMARY OF THE INVENTION

In an exemplary embodiment of the present invention, there is disclosed a method of providing a uniform standard for everyone to compare their low crouch duck walking ability or for determining when a person has reached a minimum level of physical fitness comprising:
assuming a crouch down position;
duck walking along a course that is at least about 100 yards in length;
duck walking from start to finish along the course without standing up; and
duck walking from start to finish along the course without touching the ground with one's hands.

The more important features of the invention have thus been outlined in order that the more detailed description that follows may be better understood and in order that the present contribution to the art may better be appreciated. Additional features of the invention will be described hereinafter and will form the subject matter of the claims that follow.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

The foregoing has outlined, rather broadly, the preferred feature of the present invention so that those skilled in the art may better understand the detailed description of the invention that follows. Additional features of the invention will be described hereinafter that form the subject of the claims of the invention. Those skilled in the art should appreciate that they can readily use the disclosed conception and specific embodiment as a basis for designing or modifying other structures for carrying out the same purposes of the present invention and that such other structures do not depart from the spirit and scope of the invention in its broadest form.

### BRIEF DESCRIPTION OF THE DRAWINGS

Other aspects, features, and advantages of the present invention will become more fully apparent from the following detailed description, the appended claim, and the accompanying drawings in which similar elements are given similar reference numerals.

FIG. 1 is a view of a person in a low crouch duck walk position where the person's buttocks are resting on his/hers heels at the beginning of a test in accordance with the principles of the invention;

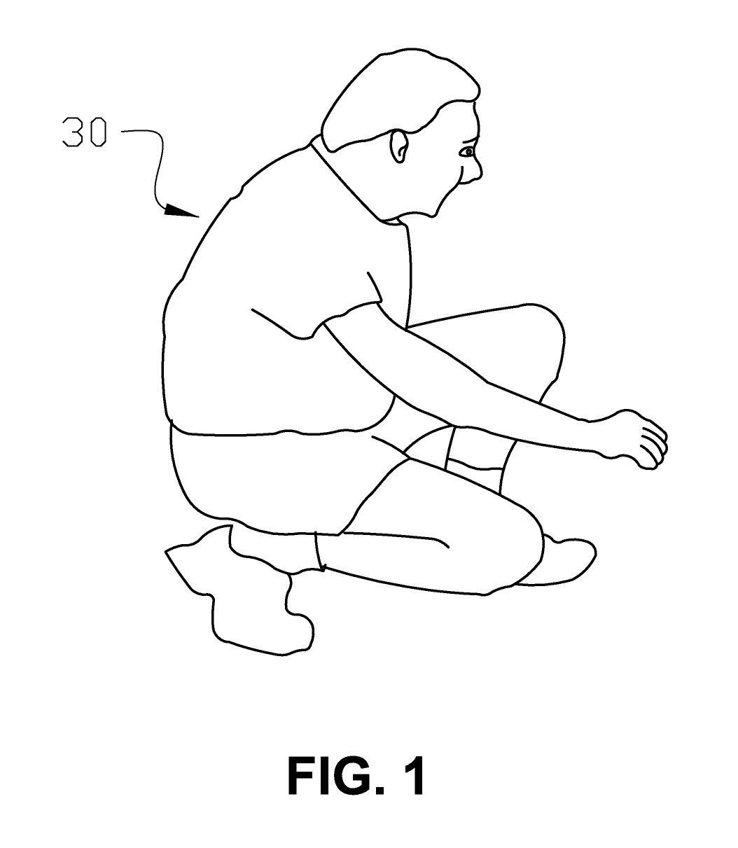

FIG. 2 is a view of the person moving forward to a finish line of about one hundred yards, more or less without standing up or touching the ground with his/her hands; and

FIG. 3 is a flow diagram of a method of determining when a person has reached a minimum level of physical fitness.

### DESCRIPTION OF THE PREFERRED EMBODIMENT

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated devices, and such further applications of the principles of the invention as illustrated therein being contemplated as would normally occur to one skilled in the art to which the invention relates.

Good health and physical fitness are generally associated with each other and various books on exercise and diet programs have been written to help individuals improve their physical fitness. Some books involve eating programs which control a person's caloric or carbohydrate intake, and others are directed toward an exercise that raise a person's heart rate to a predetermined maximum rate for a predetermined time period. Almost all require apparatus in the form of an exercise machine and/or at least one electronic sensor.

What is needed is a method which is simple to use and does not require any apparatus or equipment for determining a person's minimum physical fitness level that works for all body types.

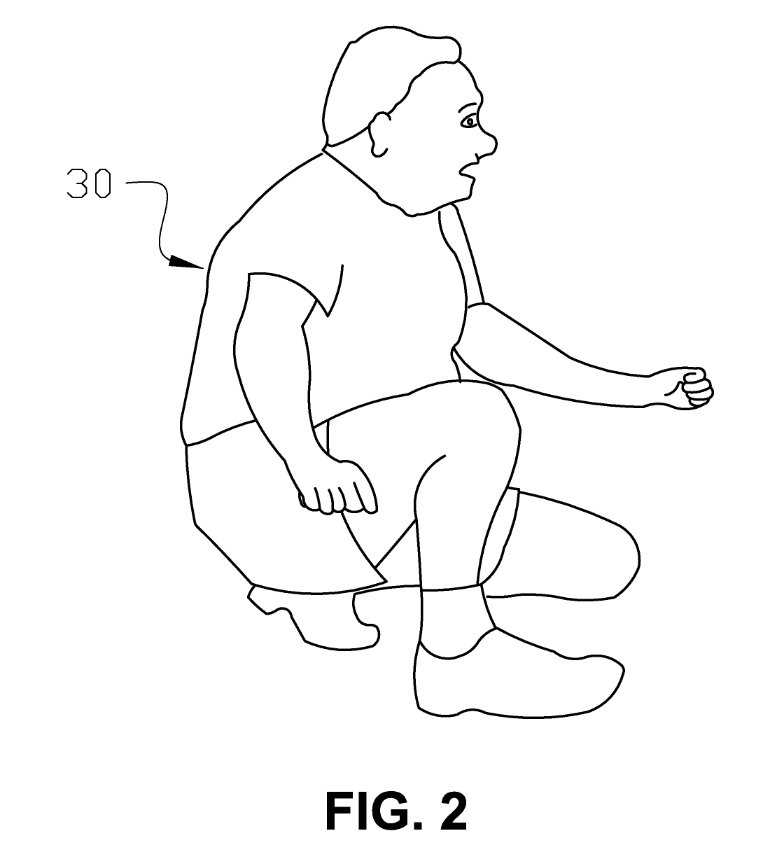

Referring to FIG, 1, there is shown a view of a person 30 in a low crouch duck walk position where the person's buttocks are resting on his/hers heels at the beginning of an exercise to determine a person's minimum level of physical fitness in accordance with the principles of the invention. FIG. 2 is a view of the person 30 moving forward toward a finish line of about one hundred yards, more or less, without standing up or touching the ground with his/her hands.

The method of determining a person's minimum level of physical fitness here disclosed relates to a one hundred yard non-stop low crouch dick walking exercise without standing up or touching the ground with one's hands.

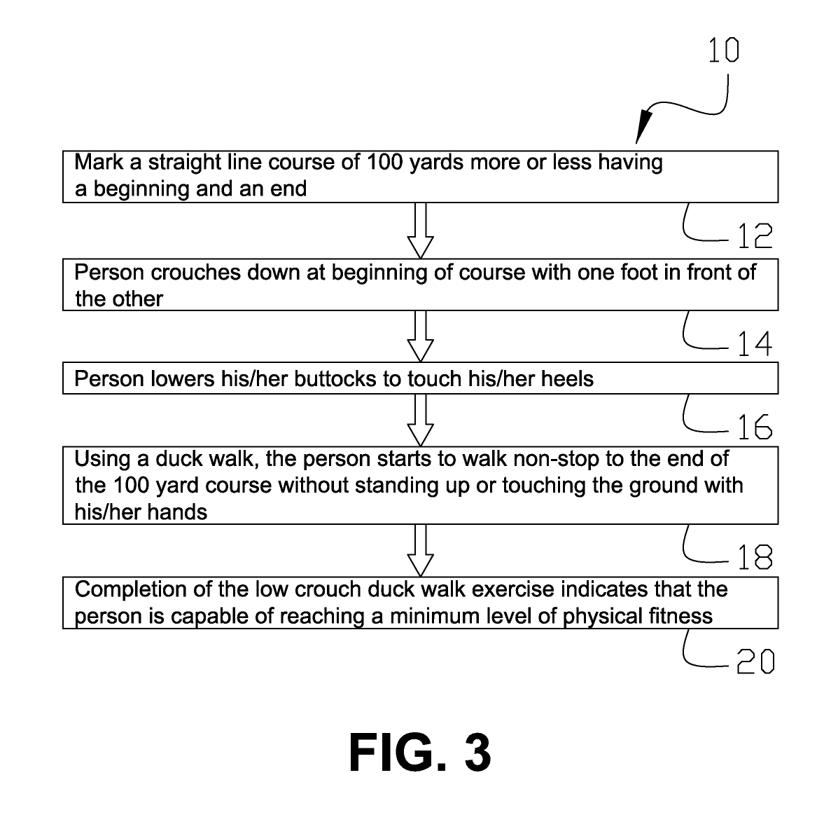

Referring to FIG. 3, there is shown a flow diagram 10 of a method for determining when a person has reached a minimum level of physical fitness. At the start, the person marks a straight line course of 100 yards, more or less, which has a beginning and an end, block 12. Once the course has been marked, the person crouches down at the beginning with one foot in front of the other, block 14. The person now lowers his/her buttocks to touch his/her heals, block 16, and while in this position and using a duck walk, the person starts to walk non-stop to the end of the 100 yard course without standing up or touching the ground with his/her hands, block 18. Completion of the low crouch dick walk exercise by the person indicates that the person is capable of reaching a minimum level of physical fitness, block 20.

The exercise includes the steps of a person assuming a low crouch position with the buttocks resting on at least one heel; then duck walking forward to cover the 100 yard distance while not stand up or touching the ground with his/her hands. The method disclosed creates a uniform standard for everyone to compare their low crouch duck walking ability.

While there have been shown and described and pointed out the fundamental novel features of the invention as applied to the preferred embodiments, it will be understood that the foregoing is considered as illustrative only of the principles of the invention and not intended to be exhaustive or to limit the invention to the precise forms disclosed. Obvious modifications or variations are possible in light of the above teachings. The embodiments discussed were chosen and described to provide the best illustration of the principles of the invention and its practical application to enable one of ordinary skill in the art to utilize the invention in various embodiments and with various modifications as are suited to the particular use contemplated All such modifications and variations are within the scope of the invention as determined by the appended claims when interpreted in accordance with the breadth to which they are entitled.

What is claimed is:

1. A method of providing a uniform standard for everyone to compare their low crouch duck walking ability or for determining when a person has reached a minimum level of physical fitness comprising:
assuming a crouch down position;
duck walking along a course that is at least about 100 yards in length;
duck walking from start to finish along the course without standing up; and
duck walking from start to finish along the course without touching the ground with one's hands.

2. The method of claim 1 wherein the duck walking is done without stopping.

3. The method of claim 1 wherein the buttocks of the person touches his/hers heels.

4. The method of claim 1 wherein the duck walking is done without stopping and the buttocks of the person touches his/hers heels.

5. The method of claim 1 wherein when assuming the crouch down position one foot is located in front of the other foot.
